(19) United States
(12) Patent Application Publication
(10) Pub. No.: US 2004/0220224 A1
(43) Pub. Date: Nov. 4, 2004

(54) Method of treating irritable bowel syndrome

(75) Inventors: Ivan Michael Richards, Kalamazoo, MI (US); Karen Patrice Kolbasa, Schoolcraft, MI (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/823,944

(22) Filed: Apr. 13, 2004

Related U.S. Application Data
(60) Provisional application No. 60/462,921, filed on Apr. 15, 2003.

(51) Int. Cl. A61K 31/445; A61K 31/40; A61K 31/14; A61K 31/205; A61K 31/55

(52) U.S. Cl. 514/317; 514/408; 514/643; 514/554; 514/217.12

(57) ABSTRACT

The invention features a method of treating irritable bowel syndrome (IBS) by administering quarternary ammonium compounds of formulae I-V, described herein.

### RELATED APPLICATIONS

This application claims benefit of priority under 35 U.S.C. § 119(e) to U.S. Provisional Application No. 60/462,921, filed Apr. 15, 2003, which is incorporated herein in its entirety by reference.

### BACKGROUND

The invention relates to methods of treating irritable bowel syndrome using quarternary ammonium compounds.

Irritable Bowel Syndrome (IBS) is a functional gastrointestinal disorder. IBS is often misdiagnosed or misnamed as colitis, mucous colitis, spastic colon, irritable bowel disease or spastic bowel (colon). These misnomers persist, even though IBS is now a recognized and treatable condition. Affecting between 25 and 55 million people in the United States, IBS results in 2.5 to 3.5 million yearly visits to physicians. 20 to 40 percent of all visits to gastroenterologists are due to symptoms of IBS.

IBS is a difficult disease to diagnose because most patients suffering from IBS do not exhibit physical defects which can be observed during an examination. As a result, patients suffering from IBS are diagnosed only after all other possible digestive disorders and diseases have been ruled.

Many people believe that patients suffering from IBS have gastrointestinal muscles that are exceptionally sensitive to stimuli or triggers. While they would not normally affect others, triggers such as food or stress can provoke a strong response in a person with IBS. A person who does not have IBS may have no trouble eating a salad, or drinking coffee, but a person with IBS may exhibit symptoms such as pain, bloating, and diarrhea.

The symptoms of IBS can include one or more of the following: gas, pain, bloating, nausea, vomiting, mucous in the stool, constipation, and diarrhea.

Cramps are often relieved by a bowel movement, but some people with IBS may have cramps and be unable to pass anything. Severity of symptoms can vary widely and be described as anything from a mild annoyance to debilitating. Blood in the stool, fever, weight loss, vomiting bile, and persistent pain are not symptoms of IBS and may be the result of some other problem.

IBS is second only to the common cold as being the most frequent cause of absenteeism from work and school. Many people with IBS describe that symptoms frequently occur shortly after, or even during, meals. Fatty foods, alcohol, caffeine and gas-producing foods (such as broccoli or beans) have regularly been named culprits in causing IBS attacks. It can be difficult to track down which particular foods can act as triggers for IBS.

Further complicating the issue, not every person with IBS responds with symptoms to the same foods. The range of triggers is unique to each individual, although there are may common elements among most people with IBS. Symptoms can also be intermittent. Something that was fine to eat last week may be causing symptoms today.

### SUMMARY

In general, the invention features a method of treating irritable bowel syndrome (IBS) by administering quarternary ammonium compounds.

In one aspect, the invention features a method of administering quaternary ammonium compounds of the formula I

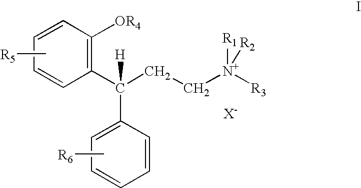

and the enantiomer thereof

wherein each R₁, R₂, and R₃ is independently H, C₁-C₅ alkyl optionally substituted with phenyl, or C₂-C₆ alkenyl, or wherein two of R₁, R₂ and R₃ may form a ring together with the quaternary ammonium nitrogen.

where R₄ is

—H,

—CO—R₄₋₁ where R₄₋₁ is

C₁-C₄ alkyl,

C₁-C₄ alkoxy,

—NR₄₋₂R₄₋₃ where R₄₋₂ and R₄₋₃ are the same or different and are —H or C₁-C₄ alkyl,

where R₅ and R₆ are the same or different and are

—H,

C₁-C₄ alkyl optionally substituted with 1 or 2

—OH,

C₁-C₄ alkoxy,

—COOH,

—CO—O—(C₁-C₃ alkoxy)

—F, —Cl, Br,

—CF₃,

where X⁻ is selected from the group consisting of the anions of the following acids hydrochloric, hydrobromic, hydroiodic, sulfuric, phosphoric, nitric, citric, methanesulfonic CH₃—(CH₂)ₙ₁—COOH where n₁ is 0 thru 4, HOOC—(CH₂)n₁—COOH where n is as defined above, HOOC—CH═CH—COOH, φ-COOH.

In another aspect, the invention features a method of administering quaternary ammonium compounds of formula II

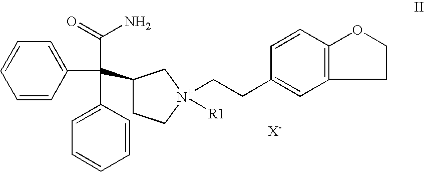

and any stereoisomers thereof, wherein

R₁ is selected from C₁-C₆ alkyl, —CH₂—(C₁-C₄ alkenyl), and —CH₂—(C₁-C₆ alkynyl), each of which is optionally substituted with a group selected from phenyl, C₁-C₄ alkoxy, and hydroxyl; and

X represents an anion of a pharmaceutically acceptable acid.

In another aspect, the invention features a method of administering quaternary ammonium compounds of formula III

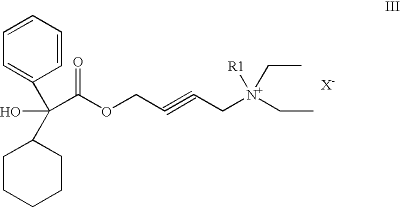

and any stereoisomers thereof, wherein

R₁ is selected from C₁-C₆ alkyl, —CH₂—(C₁-C₄ alkenyl), and —CH₂—(C₁-C₆ alkynyl), each of which is optionally substituted with a group selected from phenyl, C₁-C₄ alkoxy, and hydroxyl; and

X represents an anion of a pharmaceutically acceptable acid.

In still another aspect, the invention features a method of administering quaternary ammonium compounds of formula IV

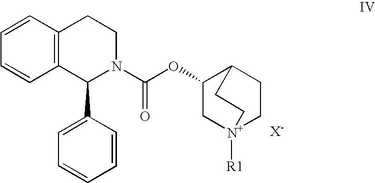

and any stereoisomers thereof, wherein

R₁ is selected from C₁-C₆ alkyl, —CH₂—(C₁-C₄ alkenyl), and —CH₂—(C₁-C₆ alkynyl), each of which is optionally substituted with a group selected from phenyl, C₁-C₄ alkoxy, and hydroxyl; and

X represents an anion of a pharmaceutically acceptable acid.

In still another aspect, the invention features a method of administering quaternary ammonium compounds of formula V

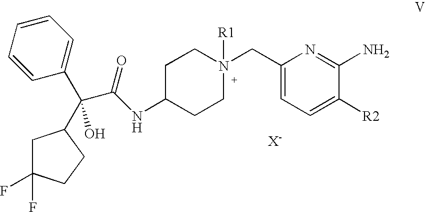

and any stereoisomers thereof, wherein

R₁ is selected from C₁-C₆ alkyl, —CH₂—(C₁-C₄ alkenyl), and —CH₂—(C₁-C₆ alkynyl), each of which is optionally substituted with a group selected from phenyl, C₁-C₄ alkoxy, and hydroxyl;

R₂ is selected from H or OH; and

X represents an anion of a pharmaceutically acceptable acid.

Embodiments of the invention may include one or more of the following. X is selected from the group consisting of the anions of the following acids: tartaric, hydrochloric, hydrobromic, hydroiodic, sulfuric, phosphoric, nitric, citric, methanesulfonic, CH₃—(CH₂)ₙ—COOH where n is 0-4, HOOC—(CH₂)ₙ—COOH where n is 1-4, HOOC—CH═CH—COOH, and benzoic. X is selected from the group consisting of iodide, bromide, and chloride.

Advantageously, the quarternary ammonium compounds of formulae I-V are antimuscarinic agents which exhibit low systemic absorption. When taken orally, the quarternary compounds exhibit an increased residence time in the gut relative to non-quarternized forms of the compounds. The prolonged residence time in the gut permits the antimuscarinic agent compounds to alleviate gut cramping, spasms, and contractions associated with IBS over a longer treatment period with less frequent dosing compared to other IBS therapies using non-quarternized forms of the compounds. The quartnerary ammonium compounds of formulae I-V inhibit gut motility by at least about 10%. For instance the compounds of formulae I-V inhibit gut motility by about 20% or even by about 30%.

### BRIEF DESCRIPTION OF THE DRAWINGS

FIG. 1 illustrates the effect of the compound of Example 3 on Intestinal Transit in Mice.

FIG. 2 illustrates the effect of the compound of Example 27 on Intestinal Transit in Mice.

FIG. 3 illustrates the Effect of non-quarternized form of the compound of Example 27 on Intestinal Transit in Mice.

### DESCRIPTION OF THE INVENTION

In describing the preferred embodiment, certain terminology will be utilized for the sake of clarity. Such terminology is intended to encompass the recited embodiments, as well as all technical equivalents that operate in a similar manner for a similar purpose to achieve a similar result. To the extent that any pharmaceutically active compound is disclosed or claimed, it is expressly intended to include all active metabolites produced in vivo, and, is expressly intended to include all enantiomers, isomers or tautomers where the compound is capable of being present in its enantiomeric, isomeric or tautomeric form. All stereoisomers have useful activity. Therefore, the invention includes use of each stereoisomer separately, as well as mixtures thereof.

The compounds of formulae I-V can be prepared by one skilled in the art. The quaternary ammonium compounds of formulae I-V may be prepared by means, well known to those skilled in the art, for preparing quaternary ammonium compounds from tertiary amines. For instance, the quaternary ammonium compounds may be produced by alkylating the tertiary nitrogen using the tertiary amines of U.S. Pat. Nos. 5,096,890, 5,973,182, 5,382,600, WO98/29402, of European Patent No. 0801067 A1, U.S. Patent Application No. 2001/0051727A1, and 5,382,600, the contents of which are hereby incorporated by reference, and other known compounds as starting materials.

The general term “quaternary ammonium compound” relates to any compound that can be regarded as derived from ammonium hydroxide or an ammonium salt by replacement of all four hydrogen atoms of the NH₄-ion by organic groups. The specific compounds are for nomenclature reasons (see e.g. Chemical Abstracts) named as “aminium” compounds, but it is possible to use the term “ammonium” in the names. For example, (3R)-3-(2-hydroxy-s-methylphenyl)-N,N-diisopropyl-N-methyl3phenylpropanl . . . aminium bromide can also be named as an ammonium compound: (3R)-[3-(2-hydroxy-s-methylphenyl)-3-phenylpropyl]diisopropylmethylammonium bromide.

By way of example, a tertiary amine according to U.S. Pat. No. 5,096,890, or its salt, is dissolved in a suitable solvent. The tertiary amine is allowed to react with an organic substrate, e.g. an organic halide. The substrate contains a C₁-C₆ alkyl, preferably a C₁-C₃ alkyl, optionally substituted with phenyl, and a leaving group. The identity of the leaving group is not critical, but it is preferred that the leaving group is a halide, such as iodide or bromide. Thus, exemplary substrates include methyl iodide, methyl bromide, ethyl iodide, propyl iodide, benzyl bromide or benzyl iodide. The resulting reaction product is a quaternary ammonium compound, which is readily crystallized in suitable solvents, known to those skilled in the art. The crystals thus produced are quaternary ammonium salts. Their identity is confirmed by standard methods, such as melting point determination, nuclear magnetic resonance (NMR) analysis and mass spectrometry.

The compounds of the invention are preferably administered as quarternary ammonium salts which include counter ions. X represents the anion, e.g., the counter ion, of a pharmaceutically acceptable acid. For instance X may be selected from the following anions: tartrate, chloride, bromide, iodide, sulfate, phosphate(s), nitrate, citrate, methanesulfonate, carboxylates with from two to six carbon atoms, dicarboxylates with from two to six carbon atoms, maleate, fumarate, and benzoate. For other acceptable quarternary ammonium salts, see Int. J. Pharm., 33, 201-217 (1986). Particularly preferred ions are chloride, iodide and bromide, especially bromide and iodide.

The substituent R₁ is selected from the group including C₁-C₆ alkyl, straight or branched, optionally substituted with 1-2 of phenyl or hydroxyl, or both. Thus, R₁ independently represent methyl, ethyl, propyl, isopropyl, butyl, isobutyl, pentyl, isopentyl, hexyl, or isohexyl, optionally substituted with 1-2 of phenyl or hydroxyl, or both. It is particularly preferred that R₁ represents methyl or ethyl, preferably methyl.

The compounds according to the present invention are antimuscarinic agents. “Antimuscarinic agents” refer to muscarinic receptor antagonists. Examples of known antimuscarinic agents include tolterodine, hydroxytolterodine, 2-(diisopropylamino) ethyl-1-phenylcyclopentanecarboxylate, propiverine, oxybutynin, trospium, temiverine, and ipratropium.

Propiverine is 1-methyl-4-piperidyl α,α-diphenyl-α-(n-propoxy)acetate and is disclosed in East German Patent 106,643 and in CAS 82-155841s (1975). Oxybutynin is 4-(diethylamino)-2-butynylalphaphenylcyclohexaneglycolate and is disclosed in UK Patent 940,540. Trospium is 3α-hydroxyspiro[1αH, SαH-nortropane30 8,1′pyrrolidinium]chloride benzilate and is disclosed in U.S. Pat. No. 3,480,623. Temiverine is 3S benzeneacetic acid, α-cyclohexyl-α-hydroxy-, 4-(diethylamino)-1,1-dimethyl-2-butynyl ester and is disclosed in U.S. Pat. No. 5,036,098. Ipratropium is 8-isopropylnoratropine methobromide and is disclosed in U.S. Pat. No. 3,505,337.

The compounds of formulae I-V have anti-cholinergic properties and unexpectedly exhibit prolonged activity in the gut relative to non-quarternized compounds. Thus, the compounds of formulae I-V are useful for the treatment of acetylcholine-mediated disorders. In particular, the compounds of are useful for treating IBS.

The compounds of the present invention are used to treat mammals, including man. The compounds according to the invention, in the form of free base or salts with pharmaceutically acceptable acids, or solutions thereof, can be brought into suitable dosage forms, such as compositions for administration through the oral, rectal, transdermal, parenteral, nasal, or pulmonary route in accordance with accepted pharmaceutical procedures. In particular, the compositions may be administered orally.

The compounds according to the present invention can be administered in any suitable way. The compounds according to the invention can be made up in solid or liquid form, such as tablets, capsules, powders, syrups, elixirs and the like, aerosols, sterile solutions, suspensions or emulsions, and the like. The compounds are advantageously administered orally or topically by suppository or enema.

The term “effective amount” refers to a therapeutically effective amount for treating IBS, such as to relieve cramping and gut spasms. The terms “therapy” and “therapeutically” encompass all kinds of treatments, including prophylaxis. In particular, “therapeutically effective” means that it is effective for IBS treatment.

In general, a therapeutically effective amount of antimuscarinic agent is from about 1 μg to about 1,000 μg, e.g., from about 10 μg to about 1,000 μg or from about 100 μg to about 1000 μg. However, the exact dosage of the specific compound according to the invention will vary depending on its potency, the mode of administration, the age and weight of the patient and the severity of the condition to be treated. The daily dosage may, for example, range from about 0.01 μg to about 10 μg per kg of body weight, administered singly or multiply in doses e.g. from about 1 μg to about 1,000 μg each. The compounds of formula I can be administered from one to four times daily, e.g., once or twice daily.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

The desired dose may conveniently be presented in a single dose or as divided into multiple doses administered at appropriate intervals, for example, as two, three, four or more sub-doses per day. The sub-dose itself may be further divided, e.g., into a number of discrete loosely spaced administrations.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

Formulations for oral administration may be in the form of aqueous solutions and suspensions, in addition to solid tablet and capsule formulations. The aqueous solutions and suspensions may be prepared from sterile powders or granules. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants are well and widely known in the pharmaceutical art.

Pharmaceutical compositions of the compounds of formulae I-V, or mixtures thereof, either individually or in combination with other pharmaceutical agents, may be prepared by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient.

In addition to the compound of formulae I-V, or mixtures thereof, the pharmaceutical composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutical acceptable materials. The components pharmaceutical composition can be encapsulated or tableted for convenient administration.

For oral administration, the pharmaceutical composition may be in the form of, for example, a tablet, capsule, suspension or liquid. If desired, other active ingredients may be included in the composition.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

For suppository administration, the compounds may also be formulated by mixing the agent with a suitable non-irritating excipient which is solid at room temperature but liquid at rectal temperature and therefore will melt in the rectum to release the drug. Such materials include cocoa butter, beeswax and other glycerides.

Additionally, the compounds of formula I-V, or mixtures thereof, may be delivered using a sustained-release system. Various sustained-release materials have been established and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed. The compound of formulae I-V, or mixtures thereof, may also be delivered by controlled-release formulation as may be provided in a dispersion of active compound in hydroxypropyl-methyl cellulose, or other methods known to those skilled in the art. The pharmaceutical compositions also may be part of a combination therapy. In a combination therapy, the compound of formulae I-V, or mixtures thereof, and other medicaments, such as other anti-inflammatory and pain relief agents, can be administered simultaneously or at separate intervals. When administered simultaneously the compounds of formulae I-V, or mixtures thereof, and other medicaments can be incorporated into a single pharmaceutical composition or into separate compositions, e.g., the compounds of formula I-V, or mixtures thereof, in one composition and the other medicaments in another composition. Each of these compositions may be formulated with common excipients, diluents or carriers, and compressed into tablets, or formulated elixirs or solutions. The compounds can be formulated as sustained relief dosage forms and the like.

When separately administered, therapeutically effective amounts of the compound of formulae I-V, or mixtures thereof, and the other medicaments are administered on a different schedule. One may be administered before the other as long as the time between the two administrations falls within a therapeutically effective interval. A therapeutically effective interval is a period of time beginning when one of either (a) the compound of formulae I-V, or mixtures thereof, or (b) the other medicaments is administered to a mammal and ending at the limit of the beneficial effect in the treatment of ocular infection of the combination of (a) and (b).

The compounds of formulae I-V may also be administered simultaneously or together.

Without further elaboration, it is believed that one skilled in the art can, using the preceding description, practice the present invention to its fullest extent. The foregoing detailed description is given for clearness of understanding only, and no unnecessary limitations should be understood therefrom, as modifications within the scope of the invention may become apparent to those skilled in the art.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

All temperatures are in degrees Celsius. Ether refers to diethyl ether. Physiological saline refers to a 0.9% aqueous 5 sodium chloride solution. When solvent pairs are used, the ratios of solvents used are volume/volume (v/v). When the solubility of a solid in a solvent is used the ratio of the solid to the solvent is weight/volume (wt/v).

### Definitions

All temperatures are in degrees Celsius.

Ether refers to diethyl ether.

Tolterodine refers to 2-[(1R)-3-(diisopropylamino)-1-phenylpropyl]4-methylphenol also known as N,N-diisopropyl-3-(2-hydroxy-5-methylphenyl)-3-phenylpropylamine, a compound of the formula:

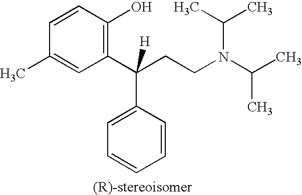

Hydroxytolterodine refers to 2-[(1R)-3-(diisopropylamino)-1-phenylpropyl]-4-(hydroxymethyl)phenol, a compound of the formula:

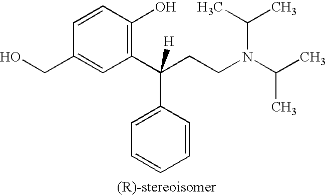

Physiological saline refers to a 0.9% aqueous sodium chloride solution.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When solvent pairs are used, the ratios of solvents used are volume/volume (v/v).

When the solubility of a solid in a solvent is used the ratio of the solid to the solvent is weight/volume (wt/v).

FEV₁ refers to Force Expiratory Volume in one second.

FEV₁/FVC refers to the ratio of the Force Expiratory Volume/Force Vital Capacity.

### Example 1

### Tolterodine Free Base

Tolterodine tartrate (2.1 g) is mixed with water (45 mL water) and toluene (2.5 mL). Sodium carbonate (800 mg) is added to the slurry. Sodium hydroxide (2.0 N, 1.5 mL) is added. The mixture is extracted three times with toluene (3 mL) saving the organic phase. Potassium carbonate is added to the organic phase to give the title compound in solution.

### Example 2

### (3R)-3-(2-Hydroxy-5-methylphenyl)-N,N-diisopropyl-N-methyl-3-phenylpropan-1-aminium iodide

Tolterodine free base (EXAMPLE 1, 0.5 M, 2.5 ml) is reacted with methyl iodide (1 ml). Acetonitrile (5 mL) is added to the mixture and stirred over night at 20-25°. The solvent is removed by blowing dry nitrogen. Acetone (1 mL) and hexane (2 ml) are added and the mixture filtered at 20-25° to give the title compound.

### Example 3

### (3R)-3-(2-Hydroxy-5-methylphenyl)-N,N-diisopropyl-N-methyl-3-phenylpropan-1-aminium bromide

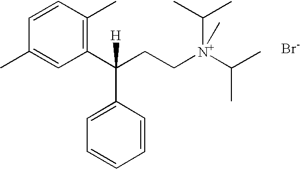

A sealed mixture of methyl bromide (100 g) and 2-[(1R)-3-(diisopropylamino)-1-phenylpropyl]-4-methylphenol (14 g) in acetone (100 mL) is stirred at 20-25° for 4 days. The mixture is cooled to −10° C. and the precipitate is filtered off and washed with ether and dried to give the title compound, mp 185°.

### Example 4

### (3R)-N-Ethyl-3-(2-hydroxy-5-methylphenyl)-N,N-diisopropyl-3-phenylpropan-1-aminium iodide

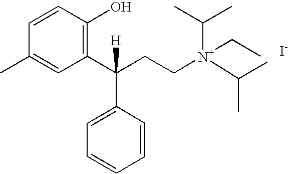

Following the general procedure of EXAMPLE 2 and making non critical variations, but starting with ethyl iodide the title compound is obtained.

### Example 5

### (3R)-3-(2-Hydroxy-5-methylphenyl)-N,N-diisopropyl-3-phenyl-N-propylpropan-1-aminium iodide

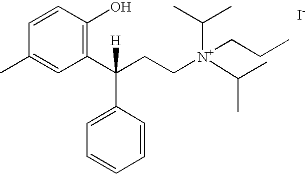

Following the general procedure of EXAMPLE 2 and making non critical variations, but starting with propyl iodide the title compound is obtained.

### Example 6

### (3R)-N-Benzyl-3-(2-hydroxy-5-methylphenyl)-N,N-diisopropyl-3-phenylpropan-1-aminium iodide

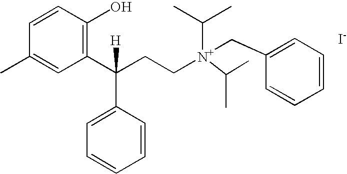

Following the general procedure of EXAMPLE 2 and making non critical variations, but starting with benzyl iodide the title compound is obtained

### Example 7

### (3R)-N-(tert-Butyl)-3-(2-hydroxy-5-methylphenyl)-N,N-dimethyl-3-phenylpropan-1-aminium bromide

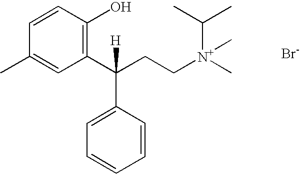

Following the general procedure of EXAMPLE 2 and making non critical variations, but starting with 2-{(1R)-3-[tert-butyl(methyl)amino]-1-phenylpropyl}-4-methylphenol the title compound is obtained.

### Example 8

### (3R)-3-[2-hydroxy-5-(hydroxymethyl)phenyl]-N,N-diisopropyl-N-methyl-3-phenylpropan-1-aminium iodide

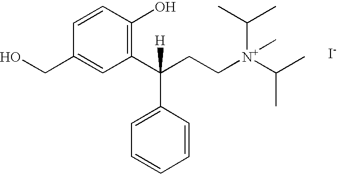

Following the general procedure of EXAMPLE 2 and making non critical variations but starting with 2-[(1R)-3-(diisopropylamino)-1-phenylpropyl]-4-(hydroxymethyl)phenol, the title compound is obtained.

### Example 9

### (3R)-3-(2-hydroxyphenyl)-N,N-diisopropyl-N-methyl-3-phenylpropan-1-aminium bromide

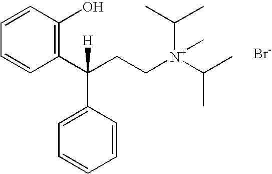

Following the general procedure of EXAMPLE 3 and making non critical variations but starting with 2-[(1R)-3-(diisopropylamino)-1-phenylpropyl]phenol, the title compound is obtained.

### Example 10

### (3S)-3-(2-hydroxyphenyl)-N,N-diisopropyl-N-methyl-3-phenylpropan-1-aminium bromide

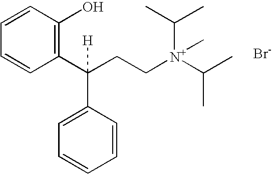

Following the general procedure of EXAMPLES 3 and making non critical variations but starting with 2-[(1S)-3-(diisopropylamino)-1-phenylpropyl]phenol the title compound is obtained.

### Example 11

### (3R)-3-(5-Chloro-2-hydroxyphenyl)-N,N-diisopropyl-N-methyl-3-phenylpropan-1-aminium bromide

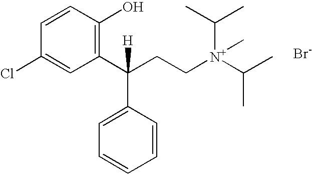

Following the general procedure of EXAMPLE 3 and making non critical variations but starting with 4-chloro-2-[(1R)-3-(diisopropylamino)-1-phenylpropyl]phenol, the title compound is obtained.

### Example 12

### (3R)-3-(5-Bromo-2-hydroxyphenyl)-N,N-diisopropyl-N-methyl-3-phenylpropan-1-aminium bromide

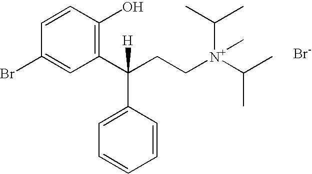

Following the general procedure of EXAMPLE 3 and making non critical variations but starting with 4-bromo-2-[(1R)-3-(diisopropylamino)-1-phenylpropyl]phenol, the title compound is obtained.

### Example 13

### (3R)-3-[2-(acetyloxy)-5-methylphenyl]-N,N-diisopropyl-N-methyl-3-phenylpropan-1-aminium iodide

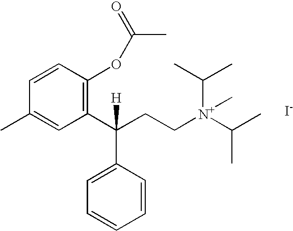

Following the general procedure of EXAMPLE 2 and making non critical variations but starting with 2-[(1R)-3-(diisopropylamino)-1-phenylpropyl]-4-methylphenyl acetate, the title compound is obtained.

### Example 14

### (3R)-3-[2-(isobutyryloxy)-5-methylphenyl]-N,N-diisopropyl-N-methyl-3-phenylpropan-1-aminium iodide

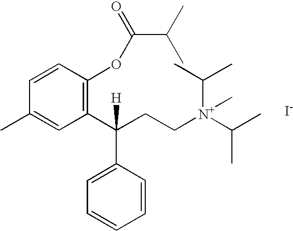

Following the general procedure of EXAMPLE 2 and making non critical variations but starting with 2-[(1R)-3-(dfisopropylamino)-1-phenylpropyl]-4-methylphenyl 2-methylpropanoate, the title compound is obtained.

### Example 15

### (3R)-3-(4-Fluorophenyl)-3-(2-hydroxy-5-methylphenyl)-N,N-diisopropyl-N-methylpropan-1-aminium bromide

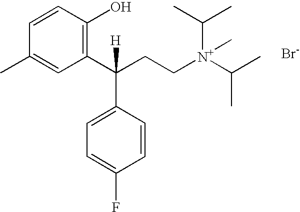

Following the general procedure of EXAMPLE 3 and making non critical variations but starting with 2-[(1R)-3-(diisopropylamino)-1-(4-fluorophenyl)propyl]-4-methylphenol, the title compound is obtained.

### Example 16

### (3R)-3-[2-hydroxy-5-(trifluoromethyl)phenyl]-N,N-diisopropyl-N-methyl-3-phenylpropan-1-aminium bromide

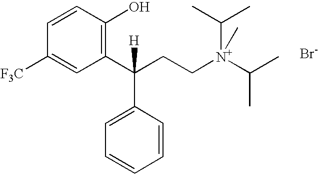

Following the general procedure of EXAMPLE 3 and making non critical variations but starting with 2-[(1R)-3-(diisopropylamino)-1-phenylpropyl]-4-(trifluoromethyl)phenol, the title compound is obtained.

### Example 17

### (3R)-3-[2-(isobutyryloxy)-5-hydroxymethylphenyl]-N,N-diisopropyl-N-methyl-3-phenylpropan-1-aminium bromide

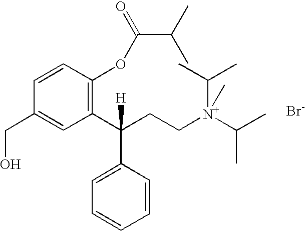

(3R)-3-[2-hydroxy-5-(hydroxymethyl)phenyl]-N,N-diisopropyl-N-methyl-3-phenylpropan-1-aminium bromide is alkylated with isobutyryl bromide to give the title compound.

### Example 18

### (3R)-3-{2-(Acetyloxy)-5-[(acetyloxy)methyl]phenyl}-N,N-diisopropyl-N-methyl-3-phenylpropan-1-aminiumbromide

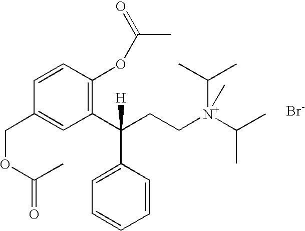

(3R)-3-[2-hydroxy-5-(hydroxymethyl)phenyl]-N,N-diisopropyl-N-methyl-3-phenylpropan-1-aminium bromide is alkylated with acetyl bromide, to give the title compound.

### Example 19

### 2-{(1R)-3-[diisopropyl(methyl)ammonio]-1-phenylpropyl}-4-methylbenzenolate

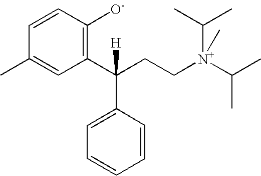

(3R)-3-(2-Hydroxy-5-methylphenyl)-N,N-diisopropyl-N-methyl-3-phenylpropan-1-aminium bromide is passed through an ion exchange column so as to remove the bromide ion and generate the title compound.

Reacting the above compound with an equivalent amount of an acid, for example, methanesulfonic acid, hydrochloric acid, acetic acid, succinic acid generates other salts of the title compound.

Reductive Amination

General Procedure A:

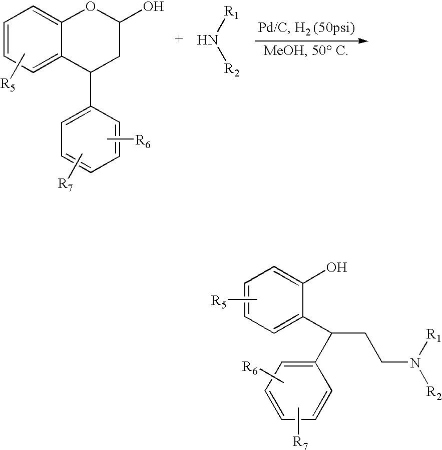

Palladium on activated carbon (1.76 g, 5% wt, Aldrich 20,568-0) was charged to a hydrogenation vessel under nitrogen followed by a MeOH (20 mL) solution of racemic lactol (4 g, 16.64 mmol) and a secondary amine (42 mmol, 2.5 equiv.). The vessel was filled with hydrogen (50 psi) and the reaction mixture was stirred vigorously at 50° C. overnight. The heterogeneous reaction mixture was filtered through celite. The resulting methanolic solution was concentrated under vacuum.

Cyclic amines, where R₁ and R₂ and the nitrogen form a ring, were obtained in after trituration with hexanes.

General Procedure B:

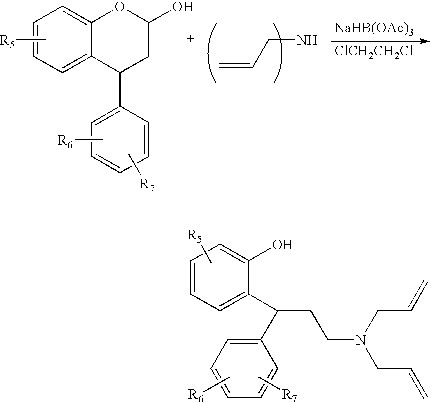

Solid NaHB(OAc)₃ (3 g, 14 mmol) was added under nitrogen to a solution of racemic lactol (2.4 g, 10 mmol) and secondary amine (0.97 g, 1.23 mL, 10 mmol) in 1,2-dichloroethane (35 mL). The reaction mixture was stirred overnight at room temperature. The reaction mixture was quenched with a saturated aqueous solution of NaHCO₃, layers were separated and the aqueous layer was extracted with ether (2×30 mL). The combined organic layers were dried other MgSO₄. After filtration, the solvents were removed under vacuum to give the crude tertiary amine as an oil. The tertiary amine obtained following this procedure was used without purification for the quaternization step.

Quaternization of the Tertiary Amines

General Procedure

Alkyl, benzyl, or allyl including a counter anion such as halide (10 equivalents) were added to a solution of free base of the tertiary amine (0.3 g, 1.02 mmol) in acetone (4 mL). The reaction mixture is stirred overnight at room temperature. The solution is concentrated to initiate the precipitation of the quaternary ammonium salt. The white precipitate is filtered, washed with diethyl ether and dried under vacuum to give the corresponding quaternized salts.

### Example 20

### 1-[3-(2-Hydroxy-5-methylphenyl)-3-phenylpropyl]-1-(2-methylprop-2-enyl)pyrrolidinium Bromide

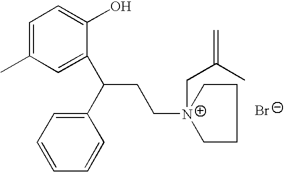

The title compound was produced by reductive amination of 6-methyl-4-phenyl-2-chromanol with pyrrolidine followed by quaternization with prop-2-enyl bromide according to the procedures described above. ¹H NMR (MeOH-d₄): δ 1.90, 2.0-2.25, 2.47-2.71, 3.21-3.31, 3.50-3.64, 3.97, 4.38, 5.36, 5.41, 6.70, 6.88, 6.95, 7.18-7.24, 7.25-7.40.

### Example 21

### 1-[3-(2-Hydroxy-5-methylphenyl)-3-phenylpropyl]-1-(3-methylbut-2-enyl)pyrrolidinium Bromide

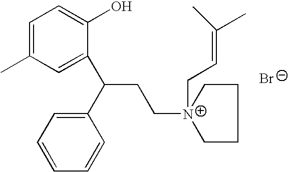

The title compound was produced by reductive amination of 6-methyl-4-phenyl-2-chromanol with pyrrolidine followed by quaternization with 3-methylbut-2-enyl bromide according to the procedures described above. ¹H NMR (MeOH-d₄): δ 1.88, 1.90, 2.0-2.25, 2.40-2.65, 3.18-3.24, 3.38-3.60, 3.97, 4.38, 5.20, 5.41, 6.68, 6.88, 6.95, 7.18-7.24, 7.25-7.40.

### Example 22

### 1-Allyl-1-[3-(2-hydroxy-5-methylphenyl)-3-phenylpropyl]pyrrolidinium Iodide

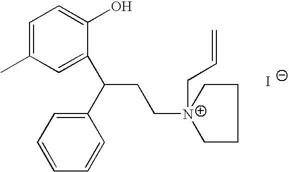

The title compound was produced by reductive amination of 6-methyl-4-phenyl-2-chromanol with pyrrolidine followed by quaternization with allyl iodide according to the procedures described above. ¹H NMR (MeOH-d₄): δ 2.0-2.25, 2.40-2.70, 3.17-3.29, 3.38-3.61, 3.97, 4.38, 5.26-5.70, 5.80-6.01, 6.68, 6.88, 6.97, 7.18-7.24, 7.25-7.40.

### Example 23

### 1-Allyl-1-[3-(2-hydroxy-5-methylphenyl)-3-phenylpropyl]pyrrolidinium Chloride

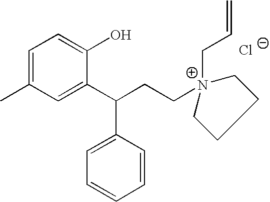

The title compound was produced via an Ion-exchange reaction. The iodide compound of Example 3 (0.6 g) was vigorously stirred with the chloride form of ion-exchange resin AG-2-X8 Bio-Rad (60 g) in 200 mL of an acetonitrile/water mixture (30/70) for 4 h. The resin was filtered on a sintered glass funnel and washed with an acetonitrile/water mixture (30/70) (40 ml). The acetonitrile was removed under vacuum and the remaining water was removed on a lyophilizer to give 0.35 g (72%) of a slightly off-white solid of the titled compound. ¹H NMR (MeOH-d₄): δ 2.0-2.25, 2.40-2.70, 3.17-3.29, 3.38-3.61, 3.97, 4.38, 5.26-5.70, 5.80-6.01, 6.68, 6.88, 6.97, 7.18-7.24, 7.25-7.40.

### Example 24

### 3-(2-Hydroxy-5-methylphenyl)-N,N-diallyl-N-methyl-3-phenyl propan-1-aminium Iodide

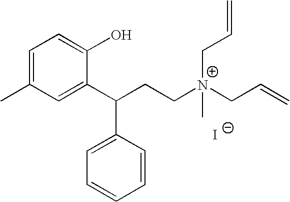

Preparation of 2-[3-(diallylamino)-1-phenylpropyl]-4-methylphenol

The tertiary amine was propuced by redcutive amination of the lactol according to the procedures described above.

Preparation of 3-(2-Hydroxy-5-methylphenyl)-N,N-diallyl-N-methyl-3-phenyl propan-1-aminium Iodide

Methyl iodide (2.2 g, 0.96 mL, 0.0155 mol) was added to a solution of the tertiary amine (0.5 g, 1.55 mmol) in a mixture of ether (3 mL) and acetone (1 mL). The reaction mixture was stirred overnight at room temperature to give a white precipitate. The white precipitate was filtered out, triturated with ether, filtered and dried under vacuum to give the title compound. ¹H NMR (MeOH-d₄): δ 2.19, 2.48-2.67, 2.98, 3.1-3.28, 3.96, 4.36, 5.61-5.7, 5.86-6.00, 6.68, 6.84, 7.01, 7.18, 7.29, 7.38.

### Example 24

### 3-(2-Hydroxy-5-methylphenyl)-N,N-diallyl-N-ethyl-3-phenylpropan-1-aminium Iodide

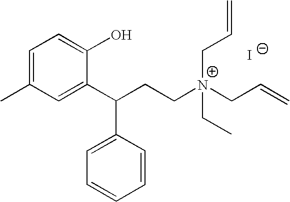

Ethyl iodide (2.42 g, 1.24 mL, 0.0155 mol) was added to a solution of 2-[3-(diallylamino)-1-phenylpropyl]-4-methylphenol (0.5 g, 1.55 mmol) in acetone (3 mL). The reaction mixture was stirred overnight at room temperature to give a white precipitate. The white precipitate was filtered then washed with ether and dried under vacuum to give The title compound. ¹H NMR (MeOH-d₄): δ 1.25, 2.19, 2.44-2.65, 3.09-3.22, 3.29-3.36, 3.91, 4.35, 5.6-5.7, 5.85-5.99, 6.8, 6.85, 7.0, 7.19, 7.30, 7.39.

### Example 25

### 1-Allyl-1-[3-(2-hydroxy-5-methylphenyl)-3-phenyl propyl]piperidinium Chloride

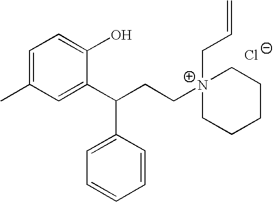

1-[3-(2-hydroxy-5-methylphenyl)-3-phenyl propyl]piperidine was prepared by reductive amination of the lactol with piperidine according to the procedures described above.

Allyl iodide (1.64 g, 0.88 mL, 0.098 mol) was added to a solution of 1-[3-(2-hydroxy-5-methylphenyl)-3-phenyl propyl]piperidine (0.3 g, 0.97 mmol) in a mixture of acetonitrile (6 mL) and methylene chloride (3 mL). The reaction mixture was stirred overnight at room temperature. The solvents were removed under vacuum and the resulting solid triturated with ether to give a solid. The solid was vigorously stirred with the chloride form of ion-exchange resin AG-2-X8 (70 g) in 200 mL of an acetonitrile/water mixture (30/70) for 4 h. The acetonitrile was removed under vacuum and the remaining water removed on a lyophilizer to give the title compound. ¹H NMR (MeOH-d₄): δ 1.64-1.83, 2.19, 2.4-2.59, 3.15-3.33, 4.0, 4.36, 5.56-5.66, 5.76-587, 6.68, 6.85, 7.19, 7.28-7.39.

### Example 26

### 3-(2-Hydroxy-5-methylphenyl)-N,N,N-triallyl-3-phenylpropan-1-aminium Bromide

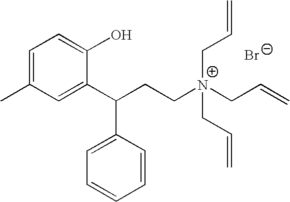

Allyl bromide (1.88 g, 1.34 mL, 0.0155 mol) was added to a solution of 2-[3-(diallylamino)-1-phenylpropyl]-4-methylphenol (0.5 g, 1.55 mmol) in acetone (3 mL). The reaction mixture was stirred overnight at room temperature to give a white precipitate. The white precipitate was filtered out, washed with ether and dried under vacuum to give the title compound. ¹H NMR (MeOH-d₄): δ 2.18, 2.47-2.67, 3.09-3.26, 3.92, 4.34, 5.64-5.70, 5.9-6.04, 6.68, 6.85, 6.92, 7.20, 7.28-7.37.

### EXAMPLE 27

### PRODUCTION OF (3S)-3-(2-amino-2-oxo-1,1-diphenylethyl)-1-[2-(2,3-dihydro-1-benzofuran-5-yl)ethyl]-1-methylpyrrolidinium iodide

(3S)-3-(2-amino-2-oxo-1,1-diphenylethyl)-1-[2-(2,3-dihydro-1-benzofuran-5-yl)ethyl]-1-pyrrolidine (1) is prepared according to the procedures described in U.S. Pat. No. 5,096,890. To COMPOUND (1), free base in toluene, is added methyl iodide (1 ml). Acetonitrile (5 ml) is added to the mixture and stirred over night at 20-25° C. The solvent is removed by blowing dry nitrogen. Acetone (1 ml) and hexane (2 ml) are added and the mixture is filtered at 20-25° C. to give the title compound. The identity of the compound has been further verified and characterized by NMR analysis, mass spectrometry, and melting point determination.

### EXAMPLE 28

### PRODUCTION OF 4-(diethylmethylaminium)-2-butynyl alpha phenyl cyclohexane glycolate iodide

4-(diethylamino)-2-butynyl alpha phenyl cyclohexane glycolate (1) is prepared according to the procedures described in U.S. Pat. No. 5,973,182. To COMPOUND (1), free base in toluene, is added methyl iodide (1 ml). Acetonitrile (5 ml) is added to the mixture and stirred over night at 20-25° C. The solvent is removed by blowing dry nitrogen. Acetone (1 ml) and hexane (2 ml) are added and the mixture is filtered at 20-25° C. to give the title compound. The identity of the compound has been further verified and characterised by NMR analysis, mass spectrometry, and melting point determination.

### EXAMPLE 29

### PRODUCTION OF 3-methyl-3-QUINUCLIDINYL 1-PHENYL-2-ISOINDOLINECARBOXYLATE

3-QUINUCLIDINYL 1-PHENYL-2-ISOINDOLINECARBOXYLATE (1) is prepared according to the procedures described in European Patent No.0801067 Al. To COMPOUND (1), free base in toluene, is added methyl iodide (1 ml). Acetonitrile (5 ml) is added to the mixture and stirred over night at 20-25° C. The solvent is removed by blowing dry nitrogen. Acetone (1 ml) and hexane (2 ml) are added and the mixture is filtered at 20-25° C. to give the title compound. The identity of the compound has been further verified and characterised by NMR analysis, mass spectrometry, and melting point determination.

### EXAMPLE 30

### PRODUCTION OF (2R)-N-[1-(6-aminopyridin-2-ylmethyl)1-methylpiperdin-4-yl]-2-[(1R)-3,3,-difluorocyclopentyl]-2-hydroxy-2-phenylacetamide iodide

(2R)-N-[1-(6-aminopyridin-2-ylmethyl)piperdin-4-yl]-2-[(1R)-3,3,-difluorocyclopentyl]-2-hydroxy-2-phenylacetamide (1) is prepared according to the procedures described in U.S. Patent Application No. 2001/0051727A1. To COMPOUND (1), free base in toluene, is added methyl iodide (1 ml). Acetonitrile (5 ml) is added to the mixture and stirred over night at 20-25° C. The solvent is removed by blowing dry nitrogen. Acetone (1 ml) and hexane (2 ml) are added and the mixture is filtered at 20-25° C. to give the title compound. The identity of the compound has been further verified and characterised by NMR analysis, mass spectrometry, and melting point determination.

### Example 31

### Inhibiting Gut Motility

Intestinal Motility Model:

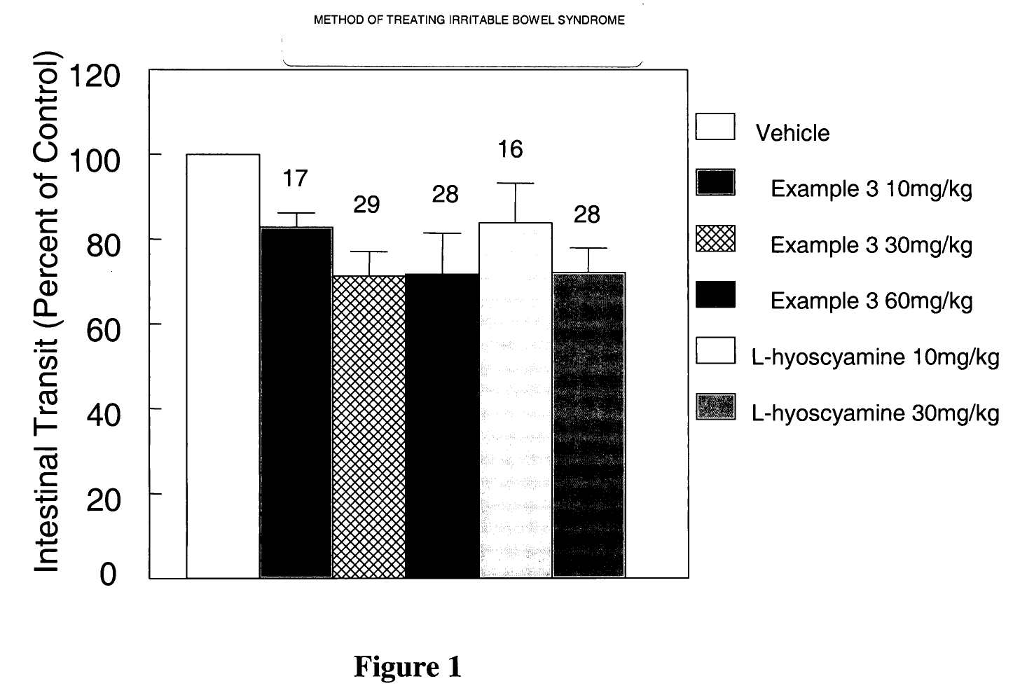

An intestinal motility model in mice may be used to evaluate of antimuscarinic compounds of formulae I-V as potential treatment for irritable bowel syndrome. In the studies, female BALB/c or CD-1 mice are fasted for 24 hours and then treated orally with one or more compounds of formulae I-V or vehicle. One hour after of treatment, the mice receive a red dye mixture via gastric gavage. Twenty minutes later, the animals are euthanized and the small intestine is removed. Gastrointestinal transit (GIT) is calculated using the measured length of the small intestine from the pyloric sphincter to the ileocecal junction and the distance traveled by the red dye front. The results are shown in FIGS. 1-3. Percent inhibition is indicated above bars.

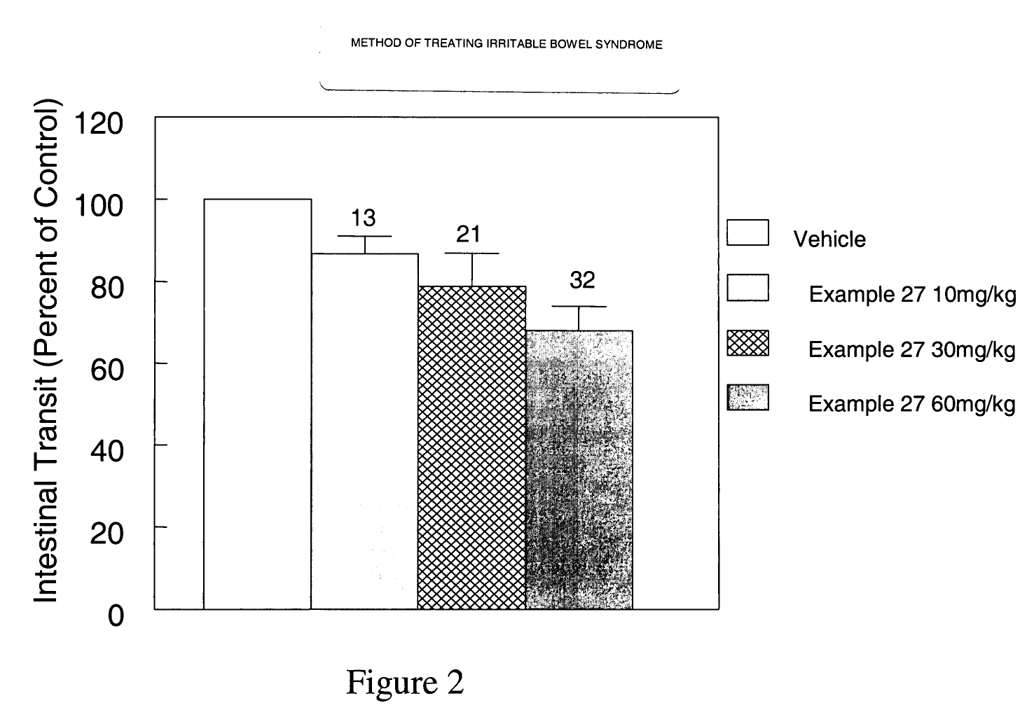

Referring to FIG. 1, the compound of Example 3, at doses of 10, 30, and 60 mg/kg, inhibited GIT 17%, 29%, and 28%, respectively. L-hyoscyamine (Levsin) was tested as a control Referring to FIG. 2, the compound of Example 27, at doses of 10, 30, and 60 mg/kg, inhibited GIT 13%, 20%, and 30%.

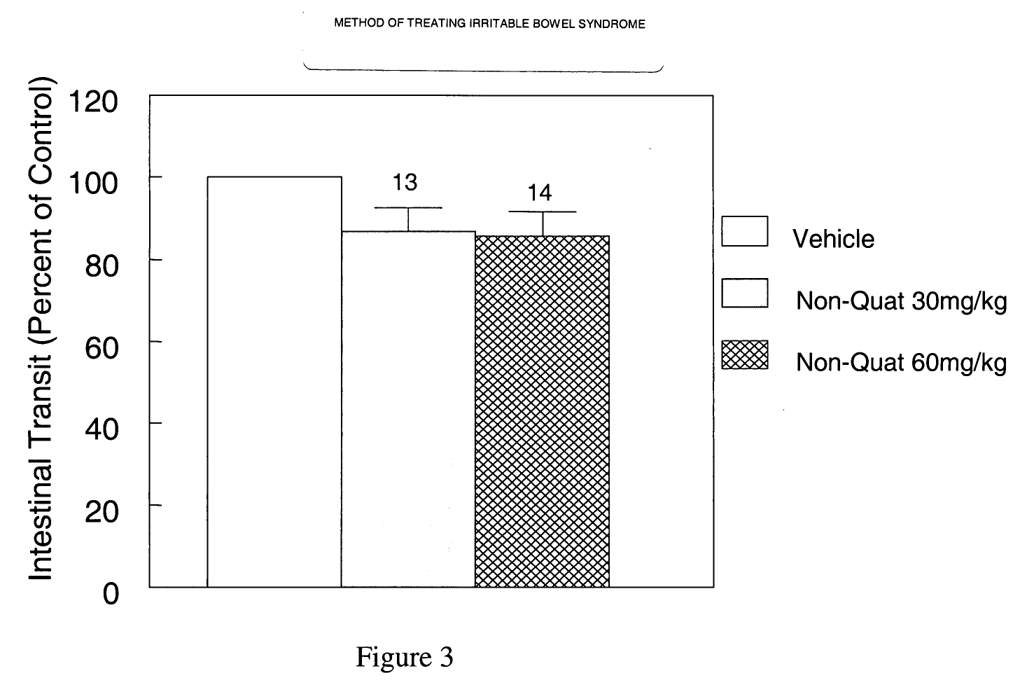

Referring to FIG. 3, a non-quarternized version of the compound of example 27, (3S)-3-(2-amino-2-oxo-1, 1-diphenylethyl)-1-[2-(2,3-dihydro-1-benzofuran-5-yl)ethyl]-1-methylpyrrolidine, at dosages of 30 mg/kg and 60 mg/kg, inhibited GIT at about 15%.

What is claimed is:

1. A method of treating IBS in a mammal, comprising administering a therapeutically acceptable amount of a compound of formula I 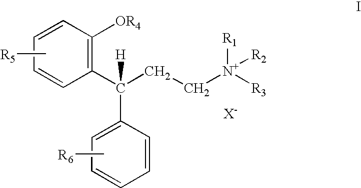
and the enantiomer thereof
wherein each R₁, R₂, and R₃ is independently H, C₁-C₅ alkyl optionally substituted with phenyl, or C₂-C₆ alkenyl, or wherein two of R₁, R₂ and R₃ may form a ring together with the quaternary ammonium nitrogen.
where R₄ is
—H,
—CO—R₄₋₁ where R₄₋₁ is
C₁-C₄ alkyl,
C₁-C₄ alkoxy,
—NR₄₋₂R₄₋₃ where R₄₋₂ and R₄₋₃ are the same or different and are —H or C₁-C₄ alkyl,
where R₅ and R₆ are the same or different and are
—H,
C₁-C₄ alkyl optionally substituted with 1 or 2
—OH,
C₁-C₄ alkoxy,
—COOH,
—CO—O—(C₁-C₃ alkoxy)
—F, —Cl, Br,
—CF₃,
where X⁻ is selected from the group consisting of the anions of the following acids hydrochloric, hydrobromic, hydroiodic, sulfuric, phosphoric, nitric, citric, methanesulfonic CH₃—(CH₂)ₙ₁—COOH where n₁ is 0 thru 4, HOOC—(CH₂)n₁—COOH where n is as defined above, HOOC—CH═CH—COOH, φ-COOH.

2. A method of treating IBS in a mammal, comprising administering a therapeutically acceptable amount of a compound of formula II 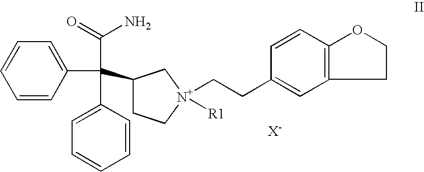
and any stereoisomers thereof, wherein
R₁ is selected from C₁-C₆ alkyl, —CH₂—(C₁-C₄ alkenyl), and —CH₂—(C₁-C₆ alkynyl), each of which is optionally substituted with a group selected from phenyl, C₁-C₄ alkoxy, and hydroxyl; and
X represents an anion of a pharmaceutically acceptable acid.

3. A method of treating IBS in a mammal, comprising administering a therapeutically acceptable amount of a compound of formula III 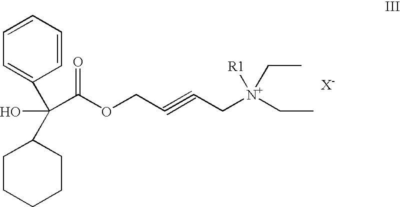
and any stereoisomers thereof, wherein
R₁ is selected from C₁-C₆ alkyl, —CH₂—(C₁-C₄ alkenyl), and —CH₂—(C₁-C₆ alkynyl), each of which is optionally substituted with a group selected from phenyl, C₁-C₄ alkoxy, and hydroxyl; and
X represents an anion of a pharmaceutically acceptable acid.

4. A method of treating IBS in a mammal, comprising administering a therapeutically acceptable amount of a compound of formula IV 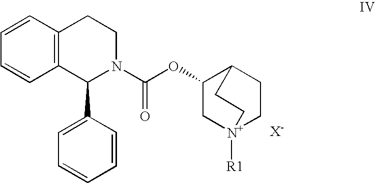
and any stereoisomers thereof, wherein
R₁ is selected from C₁-C₆ alkyl, —CH₂—(C₁-C₄ alkenyl), and —CH₂—(C₁-C₆ alkynyl), each of which is optionally substituted with a group selected from phenyl, C₁-C₄ alkoxy, and hydroxyl; and
X represents an anion of a pharmaceutically acceptable acid.

5. A method of treating IBS in a mammal, comprising administering a therapeutically acceptable amount of a compound of formula V 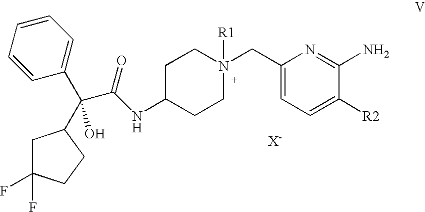
and any stereoisomers thereof, wherein
R₁ is selected from C₁-C₆ alkyl, —CH₂—(C₁-C₄ alkenyl), and —CH₂—(C₁-C₆ alkynyl), each of which is optionally substituted with a group selected from phenyl, C₁-C₄ alkoxy, and hydroxyl;
R₂ is selected from H or OH; and
X represents an anion of a pharmaceutically acceptable acid.

6. The method of any of claims 1-5, wherein X is selected from the group consisting of the anions of the following acids: tartaric, hydrochloric, hydrobromic, hydroiodic, sulfuric, phosphoric, nitric, citric, methanesulfonic, CH₃—(CH₂)ₙ—COOH where n is 0-4, HOOC—(CH₂)n—COOH where n is 1-4, HOOC—CH═CH—COOH, and benzoic.

7. The method of any of claims 1-5, wherein X is selected from the group consisting of iodide, bromide, and chloride.

8. The method of any of claims 1-5, wherein compound of formula I, II, III, IV, or V is a component of a pharmaceutical composition.

9. The method of claim 8, wherein the pharmaceutical composition comprises between about 1 mg and about 1000 mg of the compound of the formula I, II, III, IV, or V.

10. The method of claim 9, wherein the pharmaceutical composition comprises between about 200 mg and about 800 mg of the compound of the formula I, II, III, IV, or V.

11. The method of claim 9, wherein the pharmaceutical composition comprises about 600 mg of the compound of the formula I, II, III, IV, or V.

12. The method of any of claims 1-5, wherein the compound of the formula I, II, III, IV, or V.is administered orally.

13. The method of claim 12, wherein the compound of formula I, II, III, IV, or V is, a component of a tablet or capsule.

14. The method of any of claims 1-5, wherein the compound of the formula I, II, III, IV, or V.is administered as a component of a suppository.

15. The method of any of claims 1-5, wherein the compound of the formula I, II, III, IV, or V.is administered a component of an enema.

16. The method of any of claims 1-5, wherein the therapeutically effective amount of the compound of formula I, II, III, IV, or V, or mixtures thereof, are administered to a mammal in an amount from about 0.1 to about 100 mg/kg of mammal body weight/day.

17. The method of claim 16, wherein the therapeutically effective amount of the compound of formula I, II, III, IV, or V, or mixtures thereof, administered to a mammal is about 600 mg per day.

18. The method of any of claims 1-5, wherein administering the therapeutically effective amount includes administering a compound of formula I, II, III, IV, or V, or mixtures thereof, in one or more doses per day.

19. The method of any of claims 1-5, wherein the compound of formula I, II, III, IV, or V is selected from
(3R)-3-(2-Hydroxy-1-methylphenyl)-N,N-diisopropyl-N-methyl-3-phenylpropan-1-aminium iodide;
(3R)-3-(2-Hydroxy-1-methylphenyl)-N,N-diisopropyl-N-methyl-3-phenylpropan-1-aminium bromide;
(3R)-N-Ethyl-3-(2-hydroxy-5-methylphenyl)-N,N-diisopropyl-3-phenylpropan-1-aminium iodide;
(3R)-3-(2-Hydroxy-5-methylphenyl)-N,N-diisopropyl-3-phenyl-N-propylpropan-1-aminium iodide;
(3R)-N-Benzyl-3-(2-hydroxy-5-methylphenyl)-N,N-diisopropyl-3-phenylpropan-1-aminium iodide;
(3R)-N-(tert-Butyl)-3-(2-hydroxy-5-methylphenyl)-N,N-dimethyl-3-phenylpropan-1-aminium bromide;
(3R)-3-[2-hydroxy-5-(hydroxymethyl)phenyl]-N,N-diisopropyl-N-methyl-3-phenylpropan-1-aminium iodide;
(3R)-3-(2-hydroxyphenyl)-N,N-diisopropyl-N-methyl-3-phenylpropan-1-aminium bromide;
(3S)-3-(2-hydroxyphenyl)-N,N-diisopropyl-N-methyl-3-phenylpropan-1-aminium bromide;
(3R)-3-(5-Chloro-2-hydroxyphenyl)-N,N-diisopropyl-N-methyl-3-phenylpropan-1-aminium bromide;
(3R)-3-(5-Bromo-2-hydroxyphenyl)-N,N-diisopropyl-N-methyl-3-phenylpropan-1-aminium bromide;
(3R)-3-[2-(acetyloxy)-5-methylphenyl]-N,N-diisopropyl-N-methy1-3-phenylpropan-1-aminium iodide;
(3R)-3-[2-(isobutyryloxy)-5-methylphenyl]-N,N-diisopropyl-N-methyl-3-phenylpropan-1-aminium iodide;
(3R)-3-(4-Fluorophenyl)-3-(2-hydroxy-5-methylphenyl)-N,N-diisopropyl-N-methylpropan-1-aminium bromide;
(3R)-3-[2-hydroxy-5-(trifluoromethyl)phenyl]-N,N-diisopropyl-N-methyl-3-phenylpropan-1-aminium bromide;
(3R)-3-[2-(isobutyryloxy)-5-hydroxymethylphenyl]-N,N-diisopropyl-N-methyl-3-phenylpropan-1-aminium bromide;
(3R)-3-{2-(Acetyloxy)-5-[(acetyloxy)methyl]phenyl}-N,N-diisopropyl-N-methyl-3-phenylpropan-1-aminiumbromide;
2-{(1R)-3-[diisopropyl(methyl)ammonio]-1-phenylpropyl}-4-methylbenzenolate;
1-[3-(2-Hydroxy-5-methylphenyl)-3-phenylpropyl]-1-(2-methylprop-2-enyl)pyrrolidinium Bromide;
1-[3-(2-Hydroxy-5-methylphenyl)-3-phenylpropyl]-1-(3-methylbut-2-enyl)pyrrolidinium Bromide;
1-Allyl-1-[3-(2-hydroxy-5-methylphenyl)-3-phenylpropyl]pyrrolidinium Iodide;
1-Allyl-1-[3-(2-hydroxy-5-methylphenyl)-3-phenylpropyl]pyrrolidinium Chloride;
3-(2-Hydroxy-5-methylphenyl)-N,N-diallyl-N-methyl-3-phenyl propan-1-aminium Iodide;
3-(2-Hydroxy-5-methylphenyl)-N,N-diallyl-N-ethyl-3-phenylpropan-1-aminium Iodide; 1-Allyl-1-[3-(2-hydroxy-5-methylphenyl)-3-phenyl propyl]piperidinium Chloride;
3-(2-Hydroxy-5-methylphenyl)-N,N,N-triallyl-3-phenylpropan-1-aminium Bromide;
(3S)-3-(2-amino-2-oxo-1,1-diphenylethyl)-1-[2-(2,3-dihydro-1-benzofuran-5-yl)ethyl]-1-methylpyrrolidinium iodide;
4-(diethylmethylaminium)-2-butynyl alpha phenyl cyclohexane glycolate iodide;
3-methyl-3-QUINUCLIDINYL 1-PHENYL-2-ISOINDOLINECARBOXYLATE; and
(2R)-N-[1-(6-aminopyridin-2-ylmethyl)1-methylpiperdin-4-yl]-2-[(1R)-3,3,-difluorocyclopentyl]-2-hydroxy-2-phenylacetamide iodide.

20. The method of any one of claims 1-5, wherein the compound of formula I, II, III, IV, or V inhibits gut motility by at least about 10%.

21. The method of any one of claims 1-5, wherein the compound of formula I, II, III, IV, or V inhibits gut motility by at least about 20%.

22. The method of any one of claims 1-5, wherein the compound of formula I, II, III, IV, or V inhibits gut motility by at least about 30%.
